SEQUENCE LISTING (53 sequences)

SEQ ID NO: 1-9 - skipped (9 entries)

SEQ ID NO: 10 - DNA, 220 bp, Trichoderma reesei
ccgagtttacaactcccaaaccccaatgtgaacgttaccaatctgttgcctcggcgggattctctgccccgggcgcgtcgcagccccggatcccatggcgcccgccggaggaccaactcaaactcttttttctctccgtcgcggcttccgtcgcggctctgttttacctttgctctgagcctttctcggcgaccctagcgggcgtctcgaaaatgaatca

SEQ ID NO: 11 - DNA, 141 bp, Trichoderma reesei
cccaagtactatgtcaccgtcattggtatgttggcagccaacatctcattgcgtcgttgacacgtcaaactaacgatgccctcacagacgctcccggccaccgtgacttcatcaagaacatgatcactggtacttcccagg

SEQ ID NO: 12 - DNA, 755 bp, Trichoderma parareesei
attctcccttgcctatctgtccaacatttgtcgaccaaatgttgtgccgacaggtttttttcatcaccccgctttcttctacccctccgagcgacgcaaatttttttgctgccttacgatgggttttagtggggttgcatcgagcaaccccaccaatactctggccgctctgtcggatccttcgacaacagtcacctcagcacacgcgtcaccaacacagcagtctttgatccgcgatgctaaccatgttcccctccataggaagccgccgaactcggcaagggttccttcaagtacgcgtgggttcttgacaagctcaaggccgagcgtgagcgtggtatcaccatcgacattgccctctggaagttcgagactcccaagtactatgtcaccgtcattggtatgttggcagccaacatctcattgcgtcgttgacacgtcaaactaacgatgccctcacagacgctcccggccaccgtgacttcatcaagaacatgatcactggtacttcccaggccgactgcgctatcctcattatcgctgccggtactggtgagttcgaggctggtatctccaaggatggccagacccgtgagcacgctctgctcgcctacaccctgggtgtcaagcagctcatcgtcgccatcaacaagatggacactgccaactgggccgaggctcgttaccaggaaatcatcaaggagacttccaacttcatcaagaaggtcggcttcaaccccaaggccgttgctttc

SEQ ID NO: 13 - DNA, 397 bp, Trichoderma parareesei
ggggggttgtttacagggtgctgaccgagctgctctccaggacaaggacggcgatggtacgtgatggcgagtgacgcgacaacacacttattgccctctctacgaagccgcaccgaagcactttttgccgatcgatcactctctcgtcgactcgaatcatgatacatggacaagaaactgacaggcttgacctcgtaggccagatcaccaccaaggagctgggcaccgtgatgcgctctctcggccagaacccttccgagtcggagctgcaggacatgatcaacgaggttgacgccgacaacaacggttccatcgacttccctggtacgtgaattgttgggagatttggtggttgaggtacacgggctgacgtggagcggtgaagaatttctcacca

SEQ ID NO: 14 - DNA, 20 bp, Artificial Sequence
  Description of Artificial Sequence: Synthetic primer
catcgagaagttcgagaagg

SEQ ID NO: 15 - DNA, 18 bp, Artificial Sequence
  Description of Artificial Sequence: Synthetic primer
aacttgcaggcaatgtgg

SEQ ID NO: 16 - DNA, 22 bp, Artificial Sequence
  Description of Artificial Sequence: Synthetic primer
gagttcaaggaggccttctccc

SEQ ID NO: 17 - DNA, 20 bp, Artificial Sequence
  Description of Artificial Sequence: Synthetic primer
catctttctggccatcatgg

SEQ ID NO: 18-19 - skipped (2 entries)

SEQ ID NO: 20 - DNA, 180 bp, Trichoderma virens
ccgagtttacaactcccaaacccaatgtgaacgttaccaaactgttgcctcggcgggatctctgccccgggtgcgtcgcagccccggaccaaggcgcccgccggaggaccaaccaaaactcttattgtataccccctcgcgggttttttactatctgagccatctcggcgcccctcgtgg

SEQ ID NO: 21 - DNA, 614 bp, Trichoderma virens
atgggctctgtcgttctcccgcatctaaactcaggctggcacgtcgaccaggccattttatcgggttcgtcactctccttgccgttgctccatcttatttatcgtgacatgtgatgcagtgtgtgctgaccagttgcatagaagaggatcgtctcgtcgtcatccggttcggacgtgaccacgatcgggtaagaagagtcacttttactcttggatattttcccttactaactgccattaggactgcatgctgcaagatgaagtgctctacaagatagccgatcgcgtcaaaaactttgccgtcatttacctctgcgacattgacgaggtaggtttttcatctcatccatggagtaaaggccgcttttaactggaagtaggttcctgatttcaacgccatgtacgaactgtacgacccttgctctattctgttcttctttcgcaacaagcatatgatgtgcgattttggtaccggtaacaacaacaagcttaactgggtgctggaggataagcaagagctcattgacattattgaaacgatttaccgcggagcaaagaaaggtagaggtttggtggtcagccccaaggattacagcacgagacacagatactag

SEQ ID NO: 22 - protein, 143 aa, Trichoderma virens
MGSVVLPHLNSGWHVDQAILSEEDRLVVIRFGRDHDRDCMLQDEVLYKIADRVKNFAVIYLCDIDEVPDFNAMYELYDPCSILFFFRNKHMMCDFGTGNNNKLNWVLEDKQELIDIIETIYRGAKKGRGLVVSPKDYSTRHRY

SEQ ID NO: 23 - protein, 132 aa, Trichoderma virens
LPHLNSGWHVDQAILSEEDRLVVIRFGRDHDRDCMLQDEVLYKIADRVKNFAVIYLCDIDEVPDFNAMYELYDPCSILFFFRNKHMMCDFGTGNNNKLNWVLEDKQELIDIIETIYRGAKKGRGLVVSPKDY

SEQ ID NO: 24 - DNA, 21 bp, Artificial Sequence
  Description of Artificial Sequence: Synthetic primer
gaagaggatcgtctcgtcgtc

SEQ ID NO: 25 - DNA, 21 bp, Artificial Sequence
  Description of Artificial Sequence: Synthetic primer
tcaggaacctcgtcaatgtcg

SEQ ID NO: 26-29 - skipped (4 entries)

SEQ ID NO: 30 - DNA, 568 bp, Trichoderma lixii
agggatcattaccgagtttacaactcccaaacccaatgtgaaccataccaaactgttgcctcggcggggtcacgccccgggtgcgtcgcagccccggaaccaggcgcccgccggagggaccaaccaaactcttttctgtagtcccctcgcggacgttatttcttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggacctcgggagcccctaagacgggatcccggccccgaaatacagtggcggtctcgccgcagcctctcctgcgcagtagtttgcacaactcgcaccgggagcgcggcgcgtccacgtccgtaaaacacccaacttctgaaatgttgacctcggatcaggtaggaatacccgctgaacttaa

SEQ ID NO: 31 - DNA, 141 bp, Trichoderma sp.
cccaagtactatgtcaccgtcattggtatgttttcgcttttcctcattgatacttggagaccaagattctaacgtgccgctctgtagacgctcccggtcaccgtgatttcatcaagaacatgatcactggtacttcccagg

SEQ ID NO: 32-39 - skipped (8 entries)

SEQ ID NO: 40 - DNA, 564 bp, Trichoderma viride
agggatcattaccgagtttacaactcccaaacccaatgtgaacgttaccaaactgttgcctcggcggggtcacgccccgggtgcgtcgcagccccggaaccaggcgcccgccggaggaaccaaccaaactctttctgtagtcccctcgcggacgtatttctttacagctctgagcaaaaattcaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccgggggatcggcgttggggatcgggacccctcacacgggtgccggcccctaaatacagtggcggtctcgccgcagcctctcctgcgcagtagtttgcacaactcgcaccgggagcgcggcgcgtccacgtccgtaaaacacccaactttctgaaatgttgacctcggatcaggtaggaatacccgctgaacttaa

SEQ ID NO: 41 - DNA, 149 bp, Trichoderma asperellum
cccaagtactatgtcaccgtcattggtatgttttggactcttctctctagctatcgacattccaagtccgccattctaacatgctcttcccacagacgctcccggtcaccgtgatttcatcaagaacatgatcactggtacctcccagg

SEQ ID NO: 42 - DNA, 19 bp, Artificial Sequence
  Description of Artificial Sequence: Synthetic primer
tccgtaggtgaacctgcgg

SEQ ID NO: 43 - DNA, 20 bp, Artificial Sequence
  Description of Artificial Sequence: Synthetic primer
gctgcgttcttcatcgatgc

SEQ ID NO: 44 - DNA, 20 bp, Artificial Sequence
  Description of Artificial Sequence: Synthetic primer
tcctccgcttattgatatgc

SEQ ID NO: 45 - DNA, 20 bp, Artificial Sequence
  Description of Artificial Sequence: Synthetic primer
gcatcgatgaagaacgcagc

SEQ ID NO: 46 - DNA, 18 bp, Artificial Sequence
  Description of Artificial Sequence: Synthetic primer
gtgagcgtggtatcacca

SEQ ID NO: 47 - DNA, 19 bp, Artificial Sequence
  Description of Artificial Sequence: Synthetic primer
gccatccttggagaccagc

SEQ ID NO: 48-49 - skipped (2 entries)

SEQ ID NO: 50 - DNA, 545 bp, Trichoderma harzianum
  modified_base (108)..(108): a, c, t, g, unknown or other
accgaatttacaactcccaaacccaatgtgaacgttaccaaagtgttgcctcggcgggatctctgaccccgggtgcgtcgcagccccggaccaaggcgcccgccggangaccaaccaaaactcttattgtataccccctcgcgggttttttttataatctgagccttctcggcgcctctcgtaggcgtttcgaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggccctgccttggcggtggccgtttccgaaatacagtggcggtttcgccgcagcctttcctgcgcagtagtttgcacactcgcatcgggagcgcggcgcgtccacagccgttaaacacccaacttctgaaatgttgacctcggat

SEQ ID NO: 51 - DNA, 591 bp, Trichoderma harzianum
gcggagggatcattaccgagtttacaactcccaaacccaatgtgaacgttaccaaactgttgcctcggcgggatctctgccccgggtgcgtcgcagccccggaccaaggcgcccgccggaggaccaaccaaaactcttattgtataccccctcgcgggtttttttataatctgagccttctcggcgcctctcgtaggcgtttcgaaaatgaatcaaaactttcaacaacggatctcttggttctggcatcgatgaagaacgcagcgaaatgcgataagtaatgtgaattgcagaattcagtgaatcatcgaatctttgaacgcacattgcgcccgccagtattctggcgggcatgcctgtccgagcgtcatttcaaccctcgaacccctccggggggtcggcgttggggatcggccctgccttggcggtggccgtctccgaaatacagtggcggtctcgccgcagcctctcctgcgcagtagtttgcacactcgcatcgggagcgcggcgcgtccacagccgttaaacacccaacttctgaaatgttgacctcggatcaggtaggaatacccgctgaacttaagcatatca

SEQ ID NO: 52 - DNA, 968 bp, Trichoderma afroharzianum
cactggtacttcccaggccgattgcgctatcctcatcattgccgccggtactggtgagttcgaggctggtatctccaaggatggccagacccgtgagcacgctctgctcgcctacaccctgggtgttaagcagctcatcgttgccatcaacaagatggacactgccaactgggccgaggctcgttaccaggaaatcatcaaggagacttccaacttcatcaagaaggtcggcttcaaccccaaggctgttgctttcgtccccatctccggtttcaacggtgacaacatgctccagccctccaccaactgcccctggtacaagggctgggagaaggagaccaaggctggcaagttcaccggcaagaccctccttgaggccatcgactccatcgagccccccaagcgtcccacggacaagcccctccgtcttcccctccaggatgtctacaagatcggtggtattggaacagttcccgtcggccgtatcgagactggtgtcctcaagcccggtatggtcgtcactttcgctccctccaacgtcaccactgaagtcaagtccgtcgagatgcaccacgagcagctcgtcgagggtgttcccggtgacaacgttggtttcaacgtcaagaacgtttccgttaaggaaattcgccgtggtaacgttgccggtgactccaagaacgacccccccatgggtgccgcttctttcaccgctcaggtcatcgtcatgaaccaccctggccaggtcggtgccggctacgcccccgttcttgactgccacactgcccacattgcctgcaagttcgccgagctccaggagaagatcgaccgccgtaccggtaaggctaccgagactgcccccaagttcatcaagtccggtgactctgccatcgtcaagatgattccctccaagcccatgtgcgttgaggctttcaccgactaccctcccctgggtcgtttcgccgtccgtg

SEQ ID NO: 53 - DNA, 222 bp, Trichoderma longibrachiatum
ccgagtttacaactcccaaacccaatgtgaacgttaccaatctgttgcctcggcgggattctcttgccccgggcgcgtcgcagccccggatcccatggcgcccgccggaggaccaactccaaactctttttttctctccgtcgcggctcccgtcgcggctctgttttatttttgctctgagcctttctcggcgaccctagcgggcgtctcgaaaatgaatca

(19) United States
(12) Patent Application Publication
(10) Pub. No.: US 2022/0332658 A1
(43) Pub. Date: Oct. 20, 2022

(54) BIOLOGICAL CONTROL AGENT FORMULATIONS INCLUDING TRICHODERMA

(71) Applicant: Shared-X LLC, Grand Cayman (KY)

(72) Inventors: Jose Antonio de Cote, Miami, FL (US); Patrick Vasquez De Velasco, Lima (PE); Enrique Monte Vasquez, Salamanca (ES)

(21) Appl. No.: 17/659,006

(22) Filed: Apr. 12, 2022

Related U.S. Application Data
(60) Provisional application No. 63/201,117, filed on Apr. 13, 2021.

(51) Int. Cl. C05F 11/08; C05G 5/12; C05G 5/14; C12N 1/14; A01N 63/38; A01N 25/02

(52) U.S. Cl. CPC C05F 11/08; C05G 5/12; C05G 5/14; C12N 1/145; A01N 63/38; A01N 25/02

(57) ABSTRACT

The present disclosure relates to formulations including Trichoderma isolates, as well as methods thereof. In particular embodiments, the formulation can include one or more Trichoderma isolates, in which the formulation can include solid carriers and/or liquid carriers. Such formulations can be useful for treating or protecting plants, as well as counteracting growth of pathogenic organisms in proximity to the plant.

### STATEMENT OF GOVERNMENT INTEREST

This invention was made with Government support under Contract No. 1951282 awarded by the National Science Foundation. The Government has certain rights in the invention.

### INCORPORATION BY REFERENCE

An Application Data Sheet is filed concurrently with this specification as part of the present application. Each application that the present application claims benefit of or priority to as identified in the concurrently filed Application Data Sheet is incorporated by reference herein in their entireties and for all purposes.

### INCORPORATION BY REFERENCE OF SEQUENCE LISTING PROVIDED AS A TEXT FILE

The instant application contains a Sequence Listing which has been submitted electronically in ASCII format and is hereby incorporated by reference in its entirety. Said ASCII copy, created on Jun. 27, 2022, is named SHARP005US_SL.txt and is 15,788 bytes in size.

### FIELD

The present disclosure relates to formulations including one or more Trichoderma isolates, as well as methods thereof.

### BACKGROUND

Modern agricultural techniques rely on various chemicals to provide pathogen control and growth stimulation. There is an increased emphasis and awareness of the effects that such chemicals can have, not only on crop production, but also on environmental impact.

### SUMMARY

The present disclosure relates to formulations including a biological control agent (BCA), as well as methods of making and using such compositions. In particular embodiments, the formulations include one or more Trichoderma isolates, in which the formulation can include solid carriers and/or liquid carriers. Such formulations can be useful for treating or protecting plants, as well as counteracting growth of pathogenic organisms in proximity to the plant. In particular embodiments, the formulation may have potential as a biostimulant.

In a first aspect, the present disclosure encompasses a formulation including a Trichoderma parareesei isolate and a Trichoderma virens isolate. In some embodiments, the Trichoderma parareesei isolate is T. parareesei strain T6, and the Trichoderma virens isolate is T. virens strain T59.

In one embodiment, the formulation further includes an isolate of Trichoderma atroviride and/or an isolate of Trichoderma asperellum (e.g., any described herein). In particular embodiments, the isolate of Trichoderma atroviride is T. atroviride strain T11, and/or the isolate of Trichoderma asperellum is T. asperellum strain T25.

In one embodiment, the formulation further includes a liquid carrier, and the isolates include mycelium and/or spores of T. parareesei and T. virens. In some embodiments, the liquid carrier includes a mineral oil having one or more optional stabilizers.

In another embodiment, the formulation further includes a solid carrier, and the isolates include spores of T. parareesei and T. virens. In some embodiments, the solid carrier includes a water-soluble, a wettable, or a water-dispersible particle. In other embodiments, the spores are disposed on or within the particle. In particular embodiments, the particle includes a clay, a sugar alcohol, a sugar, a saccharide, or a polysaccharide. In other embodiments, the particle is a powder, a pellet, or a granule.

In a second aspect, the present disclosure encompasses a formulation including: one or more Trichoderma isolates (e.g., cellulose coated spheres); a solid carrier; and a liquid carrier. In some embodiments, the one or more Trichoderma isolates include Trichoderma parareesei, Trichoderma virens, Trichoderma atroviride, Trichoderma asperellum, or any described herein.

In other embodiments, the one or more Trichoderma isolates include a plurality of spores of Trichoderma. In some embodiments, the formulation further includes a coating surrounding one of the plurality of spores. In particular embodiments, the coating includes a polysaccharide (e.g., cellulose).

In some embodiments, the solid carrier includes a pellet. In other embodiments, the liquid carrier includes an aqueous solvent (e.g., water) or an oil.

In a third aspect, the present disclosure encompasses a binary formulation including: a first formulation (e.g., any described herein including one or more Trichoderma isolates); and a second formulation that is different than the first formulation.

In some embodiments, the second formulation includes one or more biostimulants, such as any described herein. In particular embodiments, the first and second formulations are configured to be separated (e.g., prior to delivery to a plant) and then to be combined for delivery to a plant.

In one embodiment, the first formulation includes a liquid formulation, a concentrated liquid formulation, or a solid formulation; and the second formulation includes a liquid formulation, a concentrated liquid formulation, or a solid formulation.

In a fourth aspect, the present disclosure features a method of treating a plant, the method including: preparing a composition including any formulation described herein (e.g., including one or more Trichoderma isolates) and an aqueous solvent; and delivering the composition to the plant, a portion thereof, a plant material, or a soil in proximity to the plant.

In some embodiments, the method further includes (e.g., prior to said delivering): providing the composition to an irrigation system configured to irrigate the plant.

In some embodiments, said treating includes protecting the plant against a pathogenic organism, stimulating growth of the plant, or counteracting growth of a pathogenic organism in proximity to the plant. In particular embodiments, said delivering includes delivering an effective amount of the composition for said treating.

In any embodiment herein, a concentration of the isolates (e.g., in the formulation) is from about 0.1 to 5% (w/w) of a solid formulation. In other embodiments, a concentration of each isolate (e.g., in the formulation) is from about 1×10⁷ to 1×10¹⁰ colony-forming units per gram (CFU/g) of a solid formulation.

In any embodiment herein, a concentration of the isolates (e.g., in the formulation) is from about 0.1 to 100% (w/v) or (v/v) of a liquid formulation. In other embodiments, a concentration of each isolate (e.g., in the formulation) is from about 1×10⁵ to 1×10⁸ spores per milliliter of a liquid formulation.

In any embodiment herein, the formulation further includes one or more metabolic inhibitors, stabilizers, nutrients, or additives. In other embodiments, the formulation further includes a biostimulant, wherein the biostimulant is separated from the isolates. Additional details follow.

### Definitions

As used herein, a “biological control agent” may be a biologically-derived agent, composition, or formulation that counteracts the growth of pathogenic microbes and/or that counteracts the effect of pathogenic microbes on a plant or a plant material. A non-limiting example of a biological control agent (BCA) includes a biofungicide that kills or inhibits pathogenic fungi. A BCA can also exhibit other properties, such as for a biostimulant that stimulates plant growth. Such BCAs can be derived from a biological source by, for example, culturing a microorganism and obtaining components from culture media, fermentation broth, supernatant, and like by using isolation and separation techniques. Non-limiting examples of components can include an isolate from a culture having a biological source (e.g., a microorganism, such as a bacterium, a virus, or a fungus); a spore (e.g., obtained from an isolate); mycelium (e.g., obtained from an isolate); a protein, peptide, or amino acid derived from a biological source (e.g., a microorganism); a compound derived from a biological source (e.g., a microorganism); and the like.

As used herein, a “carrier” may be solid or liquid and may include substances ordinarily employed in formulations applied to plants. Carriers may include any described herein, such as binders, encapsulating materials, carbonaceous matter, fillers, desiccants, liquids, dispersants, as well as combinations thereof. The carrier can provide a formulation that is a liquid, gel, slurry, or solid.

As used herein, an “effective amount” refers to a sufficient amount to obtain beneficial or desired result(s). An effective amount can be administered in a single operation or in several administrations. In terms of treatment or protection, a “sufficient amount” is the amount to palliate, improve, stabilize, revert, retard, or delay the progression of the stages of disease caused by a pathogen. In some embodiments, an effective amount is intended to mean an amount of a formulation described herein sufficient to inhibit the growth of a microorganism on a plant by, for example, 10%, 20%, 50%, 75%, 80%, 90%, 95%, or 1-fold, 3-fold, 5-fold, 10-fold, 20-fold, or more compared to a negative control plant not treated with a formulation or a composition provided herein.

As used herein, an “isolate” means a separated or isolated culture that includes a microorganism, as well as a portion of such a culture. Non-limiting microorganisms include a bacteria, a virus, or a fungus, as well as particular species for such microorganisms. An isolate can include one or more different components, such as spores, mycelium, proteins, nutrients, as well as combinations thereof. Isolates can be derived from a microorganism by, for example, culturing the microorganism and obtaining components from culture media (which may be a solid or a liquid), fermentation broth, supernatant, and the like by using isolation and/or separation techniques. The isolate can include components from the same microorganism, from different species of a microorganism, or from different microorganisms. As described herein, a non-limiting example of an isolate includes a Trichoderma isolate. Such a Trichoderma isolate can include one or more components obtained from a culture including one or more Trichoderma species; or obtained by combining components from two or more cultures, in which at least one culture includes one or more Trichoderma species.

As used herein, the term “mycelium” refers to a network of fungal threads or hyphae. Mycelium can be generated in a liquid culture medium or in a solid culture medium (e.g., such as by using spores).

As used herein, the term “shelf life” refers to a time period in which a formulation or a composition may be stored in a specific temperature condition without considerable loss of the attributes related to its efficacy. For storage in non-hermetic packages, storage relative humidity should be considered. In one embodiment, the minimum desirable shelf life is about 2 to 6 months at temperatures close to 40° C. In another embodiments, the shelf life is about 9 to 12 months at a refrigerated temperature close to about 4° C.; and/or about 2 to 6 months at a room temperature close to about 20° C. and less than about 25° C. In particular embodiments, conditions to avoid include freezing temperatures (e.g., about 0° C., about −18° C., or lower), very high temperatures (e.g., about 30° C., about 32° C., about 35° C., or higher), and/or high humidity (e.g., greater than about 30%, 40%, or 50% relative humidity). Viability is an attribute commonly used by pathologists to refer to the conidial quality and should preferably be greater than 80%. In particular embodiments, shelf life can be determined by the time required for the viability to be reduced to 80% at a determined temperature.

As used herein, the terms “spore” and “microbial spore” refer to a microorganism in its dormant, protected state. Fungi commonly produce unicellular spores, as a result of sexual or asexual reproduction. Spores may germinate and develop into a sporeling. Spores may survive for extended periods, often in unfavorable conditions.

The terms “polynucleotide” and “nucleic acid,” used interchangeably herein, refer to a polymeric form of nucleotides of any length, either ribonucleotides or deoxyribonucleotides. Thus, this term includes, but is not limited to, single-stranded (e.g., sense or antisense), double-stranded, or multi-stranded ribonucleic acids (RNAs), deoxyribonucleic acids (DNAs), threose nucleic acids (TNAs), glycol nucleic acids (GNAs), peptide nucleic acids (PNAs), locked nucleic acids (LNAs), or hybrids thereof, genomic DNA, cDNA, DNA-RNA hybrids, or a polymer comprising purine and pyrimidine bases or other natural, chemically or biochemically modified, non-natural, or derivatized nucleotide bases. Polynucleotides can have any useful two-dimensional or three-dimensional structure or motif, such as regions including one or more duplex, triplex, quadruplex, hairpin, and/or pseudoknot structures or motifs.

As used herein, when a polypeptide or nucleic acid sequence is referred to as having “at least X % sequence identity” to a reference sequence, it is meant that at least X percent of the amino acids or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith T F et al., J. Mol. Biol. 1981; 147:195-7) and BLAST (Basic Local Alignment Search Tool; Altschul S F et al., J. Mol. Biol. 1990; 215:403-10). These and other alignment algorithms are accessible using publicly available computer software such as “Best Fit” (Smith T F et al., Adv. Appl. Math. 1981; 2(4):482-9) as incorporated into GeneMatcher Plus™ (Schwarz and Dayhof, “Atlas of Protein Sequence and Structure,” ed. Dayhoff, M. O., pp. 353-358, 1979), BLAST, BLAST-2, BLAST-P, BLAST-N, BLAST-X, WU-BLAST-2, ALIGN, ALIGN-2, CLUSTAL, T-COFFEE, MUSCLE, MAFFT, or Megalign (DNASTAR). In addition, those skilled in the art can determine appropriate parameters for measuring alignment, including any algorithms needed to achieve optimal alignment over the length of the sequences being compared. In general, for polypeptides, the length of comparison sequences can be at least five amino acids, preferably 10, 20, 30, 40, 50, 60, 70, 80, 90, 100, 125, 150, 175, 200, 250, 300, 400, 500, 600, 700, or more amino acids, up to the entire length of the polypeptide. For nucleic acids, the length of comparison sequences can generally be at least 10, 20, 30, 40, 50, 60, 70, 80, 90, 100, 125, 150, 175, 200, 250, 300, 400, 500, 600, 700, 800, 900, 1000, 1100, 1200, 1300, 1400, 1500, 1600, 1700, 1800, 1900, 2000, 2100, or more nucleotides, up to the entire length of the nucleic acid molecule. It is understood that for the purposes of determining sequence identity when comparing a DNA sequence to an RNA sequence, a thymine nucleotide is equivalent to an uracil nucleotide.

By “substantial identity” or “substantially identical” is meant a polypeptide or nucleic acid sequence that has the same polypeptide or nucleic acid sequence, respectively, as a reference sequence, or has a specified percentage of amino acid residues or nucleotides, respectively, that are the same at the corresponding location within a reference sequence when the two sequences are optimally aligned. For example, an amino acid sequence that is “substantially identical” to a reference sequence has at least about 50%, 60%, 70%, 75%, 80%, 85%, 90%, 95%, 96%, 97%, 98%, 99%, or 100% sequence identity to the reference amino acid sequence. For polypeptides, the length of comparison sequences will generally be at least 5, 6, 7, 8, 9, 10, 11, 12, 13, 14, 15, 16, 17, 18, 19, 20, 25, 50, 75, 90, 100, 150, 200, 250, 300, or 350 contiguous amino acids (e.g., a full-length sequence). For nucleic acids, the length of comparison sequences will generally be at least 5, 10, 11, 12, 13, 14, 15, 16, 17, 18, 19, 20, 21, 22, 23, 24, or 25 contiguous nucleotides (e.g., the full-length nucleotide sequence). Sequence identity may be measured using sequence analysis software on the default setting (e.g., Sequence Analysis Software Package of the Genetics Computer Group, University of Wisconsin Biotechnology Center, 1710 University Avenue, Madison, Wis., 53705). Such software may match similar sequences by assigning degrees of homology to various substitutions, deletions, and other modifications. The present disclosure encompasses a polypeptide or nucleic acid sequence that is substantially identical to any described herein.

By “protein,” “peptide,” or “polypeptide,” as used interchangeably, is meant any chain of more than two amino acids, regardless of post-translational modification (e.g., glycosylation or phosphorylation), constituting all or part of a naturally occurring polypeptide or peptide, or constituting a non-naturally occurring polypeptide or peptide, which can include coded amino acids, non-coded amino acids, modified amino acids (e.g., chemically and/or biologically modified amino acids), and/or modified backbones. Non-limiting amino acids include glycine (Gly, G), alanine (Ala, A), valine (Val, V), isoleucine (Ile, I), leucine (Leu, L), cysteine (Cys, C), methionine (Met, M), aspartic acid (Asp, D), glutamic acid (Glu, E), arginine (Arg, R), histidine (His, H), lysine (Lys, K), asparagine (Asn, N), glutamine (Gln, Q), serine (Ser, S), threonine (Thr, T), proline (Pro, P), phenylalanine (Phe, F), tyrosine (Tyr, Y), tryptophan (Trp, W), selenocysteine (Sec, U), and pyrrolysine (Pyl, O).

The term “fragment” is meant a portion of a nucleic acid or a polypeptide that is at least one nucleotide or one amino acid shorter than the reference sequence. This portion contains, preferably, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, 1000, 1250, 1500, 1750, 1800 or more nucleotides; or 10, 20, 30, 40, 50, 60, 70, 80, 90, 100, 150, 200, 250, 300, 350, 400, 450, 500, 550, 600, 640 amino acids or more. In another example, any polypeptide fragment can include a stretch of at least about 5 (e.g., about 10, about 20, about 30, about 40, about 50, or about 100) amino acids that are at least about 40% (e.g., about 50%, about 60%, about 70%, about 80%, about 90%, about 95%, about 87%, about 98%, about 99%, or about 100%) identical to any of the sequences described herein can be utilized in accordance with the invention. In certain embodiments, a polypeptide to be utilized in accordance with the invention includes 2, 3, 4, 5, 6, 7, 8, 9, 10, or more mutations (e.g., one or more conservative amino acid substitutions, as described herein). In yet another example, any nucleic acid fragment can include a stretch of at least about 5 (e.g., about 7, about 8, about 10, about 12, about 14, about 18, about 20, about 24, about 28, about 30, or more) nucleotides that are at least about 40% (about 50%, about 60%, about 70%, about 80%, about 90%, about 95%, about 87%, about 98%, about 99%, or about 100%) identical to any of the sequences described herein can be utilized in accordance with the invention.

The term “conservative amino acid substitution” refers to the interchangeability in proteins of amino acid residues having similar side chains (e.g., of similar size, charge, and/or polarity). For example, a group of amino acids having aliphatic side chains consists of glycine (Gly, G), alanine (Ala, A), valine (Val, V), leucine (Leu, L), and isoleucine (Ile, I); a group of amino acids having aliphatic-hydroxyl side chains consists of serine (Ser, S) and threonine (Thr, T); a group of amino acids having amide containing side chains consisting of asparagine (Asn, N) and glutamine (Gln, Q); a group of amino acids having aromatic side chains consists of phenylalanine (Phe, F), tyrosine (Tyr, Y), and tryptophan (Trp, W); a group of amino acids having basic side chains consists of lysine (Lys, K), arginine (Arg, R), and histidine (His, H); a group of amino acids having acidic side chains consists of glutamic acid (Glu, E) and aspartic acid (Asp, D); a group of polar amino acids consists of D, E, N, and Q; and a group of amino acids having sulfur containing side chains consists of cysteine (Cys, C) and methionine (Met, M). Exemplary conservative amino acid substitution groups are valine-leucine-isoleucine, phenylalanine-tyrosine, lysine-arginine, alanine-valine, glycine-serine, glutamate-aspartate, and asparagine-glutamine. The present disclosure encompasses any sequence having a conservative amino acid sequence of any polypeptide sequence described herein.

### DETAILED DESCRIPTION

The present disclosure relates to formulations including one or more biological control agents, such as Trichoderma isolates. In some non-limiting examples, the formulation includes two more Trichoderma isolates. Such formulations can counteract the growth of pathogenic microbes but also exhibit other properties, such as for a biostimulant that stimulates plant growth. A biofungicide is a biological control agent that kills or inhibits pathogenic fungi; and the biological control agent can, in some instances, be a biofungicide.

The formulations herein can provide protection against phytopathogenic organisms, such as pathogenic bacteria, pathogenic fungi, and certain nematodes. In one instance, the formulations herein can protect plants by killing or inhibiting such organisms, as well as inducing resistance against pathogens, solubilizing nutrients for the plant, increasing tolerance to abiotic stress, strengthening of root systems, and/or improving plant growth. Without wishing to be limited by mechanism, such protection can include mycoparasitism, in which Trichoderma species parasitize pathogenic microbes; ecological competition, in which Trichoderma and pathogenic microbes compete for medium, nutrients, or space in the soil; action of extracellular hydrolase enzymes, such as proteases, lipases, glucanases, and the like, which can provide fungicidal activity; release of volatile metabolites, such as peptaibols, polyketides, terpenes, and the like, having various biological activity (e.g., antifungal, nematicidal, antiviral, or siderophoric activity); production of antibiotics; and root strengthening, such as by deposition of callose- or cellulose-rich barriers through Trichoderma colonization of the plant's root system.

In addition to activating plant defense pathways and inducing physical changes to the root system, Trichoderma can provide protection from fungal infection simply by outcompeting pathogenic fungi for the limited pool of nutrients. In particular embodiments, nutrient competition serves as a preventative mechanism that is most useful if the formulation is introduced during early plant development, thereby limiting the onset of pathogenic fungi.

In some embodiments, the formulation includes at least one particular Trichoderma species. In other embodiments, the formulation includes at least two particular Trichoderma species. In some cases, the species is selected from Trichoderma parareesei, Trichoderma virens, Trichoderma atroviride, and Trichoderma asperellum. Such species can be provided as isolates; and isolates can include spores, mycelium, or both. The biological state of Trichoderma can depend on the type of formulation being prepared. For instance, in some embodiments, spores are employed in a solid formulation having a solid carrier. In other embodiments, both spores and mycelium are employed in a liquid formulation having a liquid carrier. Non-limiting solid and liquid carriers are described herein.

The formulation can include a combination of multiple species, such as two, three, four, or more species. In one instance, the formulation can include T. parareesei and T. virens. The formulation can include other Trichoderma species. For instance, the formulation can further include T. atroviride (e.g., strain T11) and/or T. asperellum (e.g., strain T25). In other embodiments, the formulation can include a combination of multiple strains from the same species, such as two, three, four, or more strains. In yet other embodiments, the formulation can include a combination of multiple strains from different species, in which at least two strains or two species are different.

The relative amount of each species or strain within the formulation can provide an effective amount to provide a particular effect. Such an effective amount can be determined by the amount that will be present when delivered to the plant, a portion thereof, or soil surrounding the plant.

Isolates of T. parareesei

An isolate of the T. parareesei species can include strain T6 or strain iQB 6. In some embodiments, the species T. parareesei is characterized as CECT Accession No. 20102 (Colección Espanola de Cultivos Tipo [Spanish Type Culture Collection], Valencia, Spain), IMI No. 113135 (International Mycological Institute (IMI), now the Centre for Agriculture and Bioscience International, Wallingford, England), or IHEM Accession No. 5436 (Institute of Hygiene and Epidemiology, Mycology Laboratory (THEM), now the Belgian Co-ordinated Collections of Micro-organisms, Brussels, Belgium).

Yet other names for this species can include T. atrobrunneum F. B. Rocha, P. Chaverri & W. Jaklitsch 2014, T. aureoviride, T. aureoviride (Rifai), T. harzianum (Rifai), T. reesei, or Hypocrea jecorina.

In some instances, the species T. parareesei is defined by an intergenic sequence, such as an internal transcribed spacer (ITS) sequence. Non-limiting ITS sequences include GenBank Accession No.: AJ251698, Trichoderma reesei internal transcribed spacer 1 (ITS1), isolate 6 (SEQ ID NO:10) or a fragment thereof, as well as a nucleic acid sequence having at least 80%, 85%, 90%, or 95% sequence identity to SEQ ID NO:10 or a fragment thereof.

In other instances, the species T. parareesei is defined by the gene sequence for translation elongation factor 1-α (tef1). In one embodiment, the tef1 sequence includes GenBank Accession No.: AJ563621, Hypocrea jecorina partial tef1 gene for translation elongation factor 1, exons 5-6, strain IMI 113135 (SEQ ID NO:11) or a fragment thereof, as well as a nucleic acid sequence having at least 80%, 85%, 90%, or 95% sequence identity to SEQ ID NO:11 or a fragment thereof.

In another embodiment, the tef1 sequence includes GenBank Accession No.: KF699130, Trichoderma parareesei strain T6 translation elongation factor 1-alpha (tef1) gene, partial cds (SEQ ID NO:12) or a fragment thereof, as well as a nucleic acid sequence having at least 80%, 85%, 90%, or 95% sequence identity to SEQ ID NO:12 or a fragment thereof.

In yet another embodiment, the species T. parareesei is defined by the gene sequence for calmodulin (call), such as that provided in GenBank Accession No.: KF699131, Trichoderma parareesei strain T6 calmodulin (CAL1) gene, partial cds (SEQ ID NO:13) or a fragment thereof, as well as a nucleic acid sequence having at least 80%, 85%, 90%, or 95% sequence identity to SEQ ID NO:13 or a fragment thereof.

The presence of certain gene sequences can be determined by using primers to bind and to amplify targeted gene sequences. Non-limiting primers can include those for the fourth large intron of tef1 using primers EF1-728F (5′-CATCGAGAAGTTCGAGAAGG-3′, SEQ ID NO:14) and TEF1-LLErev (5′-AACTTGCAGGCAATGTGG-3′, SEQ ID NO:15); and those for a fragment of call using primers CAL-228F (5′-GAGTTCAAGGAGGCCTTCTCCC-3′, SEQ ID NO:16) and CAL-737R (5′-CATCTTTCTGGCCATCATGG-3′, SEQ ID NO:17). Other non-limiting methods for identifying T. parareesei are described in Rubio M B et al., “Identifying beneficial qualities of Trichoderma parareesei for plants,” Appl. Environ. Microbiol. 2014; 80(6): 1864-1873, which is incorporated herein by reference in its entirety.

Isolates of T. virens

An isolate of the T. virens species can include strain T59 or strain iQB 59. In some embodiments, the species T. virens is characterized as BioSample Accession No. SAMN00150230 (National Center for Biotechnology Information, Bethesda, Md.).

In other instances, the species T. virens is characterized by an ITS sequence including GenBank Accession No.: AJ517317, Trichoderma virens internal transcribed spacer 1 (ITS1) (SEQ ID NO:20) or a fragment thereof, as well as a nucleic acid sequence having at least 80%, 85%, 90%, or 95% sequence identity to SEQ ID NO:20 or a fragment thereof.

In yet other instances, the species T. virens is characterized by a thioredoxin-like protein gene dim1 sequence including GenBank Accession No.: FJ788527.1, Hypocrea virens strain T59 thioredoxin-like protein (Dim1) gene (SEQ ID NO:21) or a fragment thereof, as well as a nucleic acid sequence having at least 80%, 85%, 90%, or 95% sequence identity to SEQ ID NO:21 or a fragment thereof.

In yet other embodiments, the species T. virens is characterized by the presence of a thioredoxin-like protein (Dim1) or a gene expressing Dim1 (dim1), in which the Dim1 protein includes GenBank Accession No.: ACY01406.1, thioredoxin-like protein [Trichoderma virens], amino acids 1-143 (SEQ ID NO:22) or amino acids 6-137 (SEQ ID NO:23) or a fragment thereof, as well as a polypeptide sequence having at least 80%, 85%, 90%, or 95% sequence identity to one of SEQ ID NO:22, SEQ ID NO:23, or a fragment of any of these.

The presence of certain gene sequences can be determined by using primers to bind and to amplify targeted gene sequences. Non-limiting primers can include those for the dim1 gene using primers TRX-5 (5′-GAAGAGGATCGTCTCGTCGTC-3′, SEQ ID NO:24) and TRX-3 (5′-TCAGGAACCTCGTCAATGTCG-3′, SEQ ID NO:25). Other non-limiting methods for identifying T. virens are described in Moran-Diez M E et al., “TvDim1 of Trichoderma virens is involved in redox-processes and confers resistance to oxidative stresses,” Curr. Genet. 2010; 56: 63-73, which is incorporated herein by reference in its entirety.

### Other Isolates

The compositions and methods herein can include further and/or alternative isolates. In some embodiments, such isolates can also include one or more Trichoderma species.

In one embodiment, the isolate includes the species T. atroviride. Such species can include strain T11 or strain iQB 11. In particular embodiments, the species T. atroviride is characterized as CECT Accession No. 20498 or IMI No. 352941. Yet other names for this species can include T. harzianum or T. harzianum (Rifai).

In some instances, the species T. atroviride is defined by an ITS sequence, such as GenBank Accession No.: AJ224008, Trichoderma harzianum 5.8S rRNA and ITS1 and ITS2 DNA, isolate 11 (SEQ ID NO:30) or a fragment thereof, as well as a nucleic acid sequence having at least 80%, 85%, 90%, or 95% sequence identity to SEQ ID NO:30 or a fragment thereof.

In other instances, the species T. atroviride is defined by the gene sequence for tef1, such as GenBank Accession No.: AJ563609, Trichoderma cf. viride partial tef1 gene for translation elongation factor 1, exons 5-6, strain IMI 352941 (SEQ ID NO:31) or a fragment thereof, as well as a nucleic acid sequence having at least 80%, 85%, 90%, or 95% sequence identity to SEQ ID NO:31 or a fragment thereof.

In another embodiment, the isolate includes the species T. asperellum. Such species can include strain T25 or strain iQB 25. In particular embodiments, the species T. asperellum is characterized as CECT Accession No. 20178, CECT Accession No. 2941, or IMI No. 296237. Yet other names for this species can include T. viride.

In some instances, the species T. asperellum is defined by an ITS sequence, such as GenBank Accession No.: AJ223773, Trichoderma viride 5.8S rRNA gene, ITS1 and ITS2, isolate 25 (SEQ ID NO:40) or a fragment thereof, as well as a nucleic acid sequence having at least 80%, 85%, 90%, or 95% sequence identity to SEQ ID NO:40 or a fragment thereof.

In other instances, the species T. asperellum is defined by the gene sequence for tef1, such as GenBank Accession No.: AJ563611, Trichoderma asperellum partial tef1 gene for translation elongation factor 1, exons 5-6, strain IMI 296237 (SEQ ID NO:41) or a fragment thereof, as well as a nucleic acid sequence having at least 80%, 85%, 90%, or 95% sequence identity to SEQ ID NO:41 or a fragment thereof.

The presence of certain gene sequences can be determined by using primers to bind to amplify targeted gene sequences. Non-limiting primers can include those for the ITS1 region using a 5′ primer (5′-TCCGTAGGTGAACCTGCGG-3′, ITS1 primer, SEQ ID NO:42) and a 3′ primer (5′-GCTGCGTTCTTCATCGATGC-3′, ITS2 primer, SEQ ID NO:43); those for the ITS1 region, ITS2 region, and the 5.8S rDNA gene using a 5′ primer (5′-TCCGTAGGTGAACCTGCGG-3′, ITS1 primer, SEQ ID NO:42) and a 3′ primer (5′-TCCTCCGCTTATTGATATGC-3 ITS4 primer, SEQ ID NO:44); those for the 5.8S rDNA gene and the ITS2 region using a 5′ primer (5′-GCATCGATGAAGAACGCAGC-3′, ITS3 primer, SEQ ID NO:45) and a 3′ primer (5′-TCCTCCGCTTATTGATATGC-3′, ITS4 primer, SEQ ID NO:44); and those for the tef1 gene using primers tef1fw (5′-GTGAGCGTGGTATCACCA-3′, SEQ ID NO:46) and tef1rev (5′-GCCATCCTTGGAGACCAGC-3′, SEQ ID NO:47). In particular embodiments, the primer pair ITS1 (SEQ ID NO:42) and ITS4 (SEQ ID NO:44) is employed to provide amplified PCR products that range from 563 to 602 base pairs (bp), in which the products contain the ITS1 region, the ITS2 region, and the 5.8S rDNA gene and also 11 bp of the 3′ end of the 18S rDNA gene and 36 by of the 5′ end of the 28S rDNA gene.

In yet other instances, the Trichoderma species is characterized by the presence of a thioredoxin-like protein (Dim1) gene sequence including GenBank Accession No.: FJ788527.1, Hypocrea virens strain T59 thioredoxin-like protein (Dim1) gene (SEQ ID NO:21) or a fragment thereof, as well as a nucleic acid sequence having at least 80%, 85%, 90%, or 95% sequence identity to SEQ ID NO:21 or a fragment thereof. In other embodiments, the Trichoderma species is characterized by the presence of a thioredoxin-like protein (Dim1) or a gene expressing Dim1, in which the Dim1 protein includes GenBank Accession No.: ACY01406.1, thioredoxin-like protein [Trichoderma virens], amino acids 1-143 (SEQ ID NO:22) or amino acids 6-137 (SEQ ID NO:23) or a fragment thereof, as well as a polypeptide sequence having at least 80%, 85%, 90%, or 95% sequence identity to one of SEQ ID NO:22, SEQ ID NO:23, or a fragment of any of these.

Non-limiting Trichoderma species expressing Dim1, or a homolog thereof, includes T. atroviride T11 (e.g., as described herein), T. asperellum T25 (e.g., as described herein), T. harzianum T34, T. harzianum T22, and T. longibrachiatum T52.

T. harzianum T34 can be characterized as CECT Accession No. 2413 or by an ITS sequence, including GenBank Accession No.: AF278790 (SEQ ID NO:50) or a fragment thereof, as well as a nucleic acid sequence having at least 80%, 85%, 90%, or 95% sequence identity to SEQ ID NO:50 or a fragment thereof.

T. harzianum T22 can be characterized as American Type Culture Collection Accession No. 20847 (ATCC® 20847™), which has recently been identified as Trichoderma afroharzianum. T. harzianum T22 can be characterized by an ITS sequence or by the gene sequence for tef1. In one embodiment, the ITS sequence includes GenBank Accession No.: FJ545255 (SEQ ID NO:51) or a fragment thereof, as well as a nucleic acid sequence having at least 80%, 85%, 90%, or 95% sequence identity to SEQ ID NO:51 or a fragment thereof. In another embodiment, the tef1 sequence includes GenBank Accession No.: KU933430 (SEQ ID NO:52) or a fragment thereof, as well as a nucleic acid sequence having at least 80%, 85%, 90%, or 95% sequence identity to SEQ ID NO:52 or a fragment thereof.

T. longibrachiatum T52 can be characterized by an ITS sequence including GenBank Accession No.: AJ251702 (SEQ ID NO:53) or a fragment thereof, as well as a nucleic acid sequence having at least 80%, 85%, 90%, or 95% sequence identity to SEQ ID NO:53 or a fragment thereof. One or more of any of the species herein can be used alone or combined to be used together.

### Formulations and Carriers

The isolates can be provided as a liquid formulation, a solid formulation, or a semi-solid formulation. Such formulations can include one or more isolates of Trichoderma (e.g., any described herein), one or more optional carriers, and one or more optional additives.

In one embodiment, the liquid formulation includes mycelium and spores, which are, in some cases, obtained after fermentation in a bioreactor. In addition, the liquid formulation can optionally include a liquid carrier, such as a mineral oil. The liquid formulation can include any useful form, such as a concentrate (e.g., an emulsifiable concentrate) or an emulsion. In some embodiments, the liquid carrier is configured to stabilize formulation, thereby providing a shelf stable product.

In another embodiment, the solid formulation includes spores, which are disposed on or within a solid particle. The solid particle can include a water-soluble, a wettable, or a water-dispersible particle, which can be dissolved or dispersed upon addition of an aqueous solvent. The particle can be of any form, such as a powder (e.g., a wettable powder, a peat-based powder, a freeze-dried powder, a spray-dried powder, a flowable powder, and the like), a pellet, or a granule (e.g., a peat-based granule, a microgranule, and the like). Such particles can be formed of a solid carrier such as a solid phase (e.g., rice, rice kernel, rice husk, etc.), a clay (e.g., kaolin), a sugar alcohol (e.g., sorbitol), a sugar, a saccharide, or a polysaccharide (e.g., cellulose).

In one non-limiting embodiment, the solid formulation can include spores and any useful carrier. The carrier can include, for example, a mixture of kaolin, sorbitol, sugar, and water. The carrier can then be used to produce pellets, in which the size and shape of the pellets can be altered by, for instance, modifying the type and pore size of the filter. Once a pellet is obtained, it can be mixed with the spores to adhere the spore to a surface of the pellet. Such adhesion can be facilitated by the modifying or selecting carrier components having desired characteristics.

In one instance, the spore can be disposed on a solid particle. For example, the solid particle can be a rice kernel or a rice husk, in which the spore is sprayed on the rice (which serves as a core) and then dried. Any solid particle can be employed, such as a microparticle, a nanoparticle, a polymeric particle, a biodegradable particle, and the like. This solid particle, in turn, can optionally include one or more coatings. Such a coating can be formed by use of a solid carrier, such as a clay (e.g., kaolin) or a polysaccharide (e.g., cellulose). Optionally, the spore can be separated from the solid particle (e.g., a rice kernel) prior to coating with a solid carrier or adhering to a solid carrier. In such an instance, the rice kernel is employed generally as a growing medium.

In another instance, the spore can be encapsulated with a coating, thereby disposing the spore within the solid particle. The coating can have one or more layers, in which each layer can include one or more solid carriers. Useful technologies and methodologies to prepare, synthesize, and modify nanoparticles can be employed to encapsulate the spores or provide such coatings.

Each solid particle in the formulation can be same or different. For instance, the formulation can include an n number of populations of particles, in which n is more than one and in which different carriers are used in certain populations. In another instance, different Trichoderma species can be used in certain populations. For example, the formulation can include a first population of particles including a first Trichoderma species, which can be mixed with a second population of particles including a second Trichoderma species. In another example, the formulation can include an n number of populations of particles, in which each n^{th} population includes a different n^{th} Trichoderma species.

In yet another instance, the fermentation broth is filtered to provide a filter cake, which can then be extruded to form granules. During extrusion and granulation, one or more carriers and/or additives can be added. Optionally, the filtrate (e.g., the mycelium) can be lyophilized and employed (e.g., in any formulation described herein).

Any useful method can be employed to obtain the spores and/or mycelium. In one instance, culture medium is separated from the spores by use of wind current or centrifugation. Then, the spores are placed in pellets (e.g., comprising kaolin, sorbitol, and sugar).

The concentration of the Trichoderma species may depend on whether the formulation is a solid formulation or a liquid formulation. Within a solid formulation, the concentration of the isolates (e.g., spores) may be about 0.05 to 20% (w/w) of a solid formulation. In other embodiments, the concentration of the isolates (e.g., spores) may be about 1×10⁶ to 1×10¹⁵ colony-forming units per gram (CFU/g) of a solid formulation. In embodiments employing two or more Trichoderma strains, such concentrations can be for each Trichoderma species within the formulation or for the combined concentration of all Trichoderma species within the formulation. The amount of each strain within the formulation can be varied. In one instance, each strain is present various amounts (e.g., a 1:1, 1:2, or 2:1 ratio of a first Trichoderma strain and a second Trichoderma strain; or a 1:1:1:1, 1:2:1:1, or 2:2:1:1 ratio of four different Trichoderma strains). In particular embodiments, the formulation includes a 1:1 ratio of a first Trichoderma strain and a second Trichoderma strain, in which the Trichoderma strains are present at a concentration of about 1% (0.5% of the first strain and 0.5% of the second strain), and in which the carrier is present at a concentration of about 99%.

The formulation can include one or more Trichoderma isolates, and the remaining weight of the formulation can be one or more carriers (e.g., any described herein), which can optionally include any additives described herein. Non-limiting solid carriers include clays (e.g., kaolin), saccharides, polysaccharides (e.g., cellulose), and the like.

Within a liquid formulation, the concentration of the isolates (e.g., spores and/or mycelium) may be from about 0.1 to 100% (w/v) or (v/v) of a liquid formulation. In other embodiments, the concentration of the isolates is from about 1×10⁵ to 1×10¹² spores per milliliter (mL) of a liquid formulation. In yet other embodiments, the concentration of the isolates (e.g., spores) may be about 1×10⁶ to 1×10¹⁵ colony-forming units per mL (CFU/mL) of a liquid formulation. Such concentrations can be for each Trichoderma species within the formulation or for the combined concentration of all Trichoderma species within the formulation. The remaining volume of the formulation can be one or more carriers (e.g., any described herein), which can optionally include any additives described herein. Non-limiting liquid carriers include mineral oil, water, and the like.

Carriers can include any useful combination of components, such as binders, encapsulating materials, carbonaceous matter, fillers, desiccants, liquids, dispersants, and the like. Non-limiting binders can include cellulose esters (e.g., methylcellulose and hydroxypropyl cellulose), organic silicates (e.g., organosilicon esters, gums, alginate, and the like), polyalkylene oxide (e.g., polyethylene oxide), polyvinyl alcohol, polyvinyl acetate, and starch.

Non-limiting encapsulating materials include alginate, chitosan, carrageenan, cellulose, dextrin, glucan, gums (locust bean, gellan gum, xanthan gum, etc.), gelatin, whey protein, starch, vegetable or microbial gum, and combinations thereof.

Non-limiting carbonaceous matter (e.g., carbonaceous particulate solid matter) can include alginate granules, agricultural byproducts (e.g., rice), aquatic products (e.g., seaweed or kelp), barley grain, bituminous coal, leonardite (e.g., Agrolig®), muck soil activated carbon and charcoal, organic soil, peat, peat-like substances (e.g., peat moss), pyrophyllite (e.g., Pyrax®, anhydrous aluminum silicate), shale, soft coal, sphagnum moss, tree bark granules, wheat bran, wood bark compost, and combinations thereof.

Non-limiting fillers can include alginic acid, cellulose, chitin, clays, cyclodextrins, diatomaceous earths, dextrose granules or powders, gelatin, ground agricultural products, maltose-dextrose granules or powders, mineral powder (e.g., bentonite, cation clay, diatomaceous earth, kaolin, talc, and the like), porous beads or powders, porous wood products, silica, silicates, sucrose granules or powders, talc, vermiculite, zeolite, and combinations thereof. Such fillers may be useful, e.g., for improving seed coating and/or enhancing water absorption within the solid formulation.

Non-limiting desiccants include a sugar (e.g., a non-reducing sugar, a disaccharide, trehalose, sucrose, and the like), a polyol (e.g., glycerol, triethylene glycol, and the like), sugar alcohol (e.g., mannitol, sorbitol, and the like), calcium sulfate, silica, and combinations thereof.

Non-limiting liquids include water, buffer, oil (e.g., mineral oil, corn oil, olive oil, palm oil, palm kernel oil, peanut oil, rapeseed oil, rice bran oil, soybean oil, sunflower oil, vegetable oil, and the like), a non-aqueous solvent, an organic solvent (e.g., alcohol), polyethylene glycols (e.g., PEG 200, PEG 300, PEG 400, etc.), propylene glycols (e.g., PPG-9, PPG-10, PPG-17, PPG-20, PPG-26, etc.), a polyethylene glycol-polypropylene glycol copolymer, an ethoxylated alcohol, an aqueous solvent (e.g., water or a buffer), polysorbates (e.g. polysorbate 20, polysorbate 40, polysorbate 60, polysorbate 80, etc.), silicones (siloxanes, trisiloxanes, etc.), and combinations thereof. Non-limiting dispersants include alcohol ethoxylates, naphthalene sulfonates, vinylpyrrolidone polymers, nonionic surfactants, ionic surfactants such as cationic or anionic surfactants, and the like.

### Further Additives for Formulations

Such formulations can include further additives, such as metabolic inhibitors, stabilizers, nutrients, biostimulants, and the like. In one embodiment, the additive is an enzyme or a protease, which can hydrolyze proteins to provide soluble or absorptive nutrients. In another embodiment, the additive is a secondary metabolite, such as an antibiotic. In yet another embodiment, the additive is another active ingredient, such as a fungicide, an herbicide, a biosanitizer product, or a fertilizer. The type of additive(s) (e.g., fungicides, herbicides, or others) and concentration of additive(s) can be selected so that they do not negatively affect the activity or viability of the Trichoderma isolate(s).

Non-limiting metabolic inhibitors include copper sulfate. Use of such metabolic inhibitors may increase effectiveness as a fungicide.

Non-limiting stabilizers can include polysaccharides, such as cellulose; saccharides, including monosaccharides and disaccharides; sugar alcohols (e.g., mannitol or sorbitol); polyols (e.g., any described herein); starches, such as potato starch; clays, such as kaolin, and the like; organic acids, such as lactic acid, as well as combinations thereof. Use of such stabilizers may increase longevity of the spores and/or mycelium in formulations.

Non-limiting nutrients can include micronutrients, such as boron, chlorine, copper, iron, manganese, molybdenum, and zinc; macronutrients, such as calcium, magnesium, nitrogen, phosphorous, potassium, sulfur, sodium, and silicon; phosphates; nitrates (e.g., ammonium nitrate); sulfates; urea; and combinations of any of these.

Biostimulants can include any compound that can promote growth of the Trichoderma species and/or the plant. Non-limiting biostimulants can include nutrients or micronutrients (e.g., any described herein), lignosulphonates, organic acids, amino acids, carotenoids, peptides, proteins, or proteases. Non-limiting enzymes can include proteases, and the like.

Additives can be provided within the primary formulation including the Trichoderma species. For instance, such additives can include those that stabilize the formulation (e.g., for a longer shelf life at refrigerated and/or room temperature storage), increase the viability of the spores, etc. In yet other embodiments, the formulation is stored under refrigeration (e.g., about 4° C.), thereby increasing shelf life while maintaining reproducibility and viability of the spores and/or mycelium.

Alternatively, some additives may be provided at a later time but prior to delivering to a plant. For instance, such additives can include those that promote growth of the Trichoderma species, which can result in uncontrolled growth during storage. Thus, it may be preferred to separate such additives (e.g., nutrients or biostimulants) from the Trichoderma species during storage. Such additives can be provided as a separated secondary formulation, which can be combined with the primary formulation prior to delivery to a plant. In this way, the formulation can be a binary formulation include a first formulation (e.g., including one or more Trichoderma isolates) and a second formulation (e.g., including an additive, such as nutrients or biostimulants), which are configured to be separated and then combined for delivery to a plant.

In one instance, a binary formulation can be provided in a separated container having a plurality of depots, in which a first depot includes the first formulation and the second depot includes the second formulation. In use, the contents of the two depots can be combined (e.g., within the depot or in a secondary container) prior to delivery to the plant. Optionally, the contents are then incubated or fermented for a particular time frame to activate the Trichoderma isolate(s), and the activated Trichoderma isolate(s) are delivered to the plant. In another instance, the binary formulation can be provided in other useful packaging, such as separated bottles, bags (e.g., bi-laminated bags), chambers, heat sealed containers, etc.

Any of the formulations herein can be provided in packaging that provides a shelf-stable product. Non-limiting packaging include plastic bottles, bi-laminated and heat sealed bags, chambered bags, and the like. Such packaging can include characteristics that extend shelf life, such as use of ultraviolet ray blockers (e.g., dyes, coloring, or metallization for packaging), use of oxygen-free environments during packaging, etc.

### Methods of Making Formulations

The formulations herein can be prepared by combining one or more Trichoderma strains or species with an optional carrier.

An initial inoculum having the Trichoderma strain may be maintained to provide a pure strain with minimal contaminants. In some embodiments, the initial inoculum is refreshed frequently and stored at 4° C. This initial inoculum can be grown on agar to provide the starting conidia (spores), which can then be washed from the surface of the culture with sterile saline and inoculated into culture media for fermentation (e.g., in a bioreactor).

In one embodiments, concentrated quantities of Trichoderma can be obtained using a liquid medium by inoculating pure Trichoderma species onto a solid agar plate (e.g., autoclaved potato dextrose agar (PDA)) for 1 week. Conidia (spores) can be washed from the surface of the culture with sterile saline and inoculated into semi-defined balanced media (SDBM, including molasses, urea, KH₂PO₄, MgSO₄.7H₂O, and sodium citrate, pH of 7.0). Cultures can be grown at 28° C., shaking for 24 hours. The Trichoderma biomass can be filtered (e.g., through 100 μm sieves). Trichoderma produced in this way can, in some instances, possess more than 50% colonization potential through 10 days of storage. Studies have demonstrated, however, that inclusion of additives, such as additional sugars or starches, metabolic inhibitors (such as CuSO₄), or kaolin can increase the longevity of spore-containing cultures of fungus. Blended strains of Trichoderma with combinations of these additives can be prepared to assess the overall colonization potential both before and after four months of storage. In use, Trichoderma strains can be refreshed from time to time. In one instance, from a PDA plate with fresh and uncontaminated colonies, a pre-inoculum can be prepared for both liquid and solid formulations.

Any useful fermentation method can be used, such as liquid fermentation, semi-solid fermentation, solid fermentation, and biofilm formation. In one instance, fermentation can include fermentation with a culture medium including rice husk, sugar beet pulp, sugar cane bagasse, glucose, glucose nitrate, maltose, maltose peptone, hydrolyzed corn, molasses, dextrose, potato dextrose, soy, starch, whey, or other agricultural products or byproducts of the food industry in solid forms and/or broth forms. Prior to inoculation of the Trichoderma species, the culture medium can be sterilized.

Depending on the final formulation, strains may be grown at a temperature between about 24-25° C. During the period of manufactory sporulation, the ecosystem can be controlled to avoid contamination from other fungi, such as penicillium. After sufficient production in the medium, the spores can be separated (e.g., centrifugation, air separation, filtration, etc.) and stored as active matter (e.g., such as in a solid formulation including the Trichoderma isolates). Alternatively, the liquid fermentation broth can be used as the active matter (e.g., such as in a liquid formulation including the Trichoderma isolates). In another embodiment, the fermentation broth can be filtered to provide a filter cake, which can then be processed to provide a solid formulation (e.g., granules, which can be obtained by granulation of the filter cake with one or more optional carriers and/or optional additives).

In some embodiments, a solid formulation of one or more Trichoderma species can be mixed with a solid carrier. If two or more species, are employed, then various ratios of the two strains can be mixed with a solid carrier, as described herein.

Blends or combinations of Trichoderma isolates can be obtained. In one instance, each Trichoderma species is inoculated into separate bioreactors; spores and/or mycelium are obtained from each bioreactor; and then desired blends are prepared with optional carriers and/or optional additives.

Formulations can be tested in any useful manner, such as by using in vitro and in vivo assays. Non-limiting assays include colony growth inhibition assays, conidial germination inhibition assays, viability assays, quantitative growth assessment of plant growth (e.g., as determined by plant height, number and/or quality of fruit, and the like), soil sample analysis (e.g., before, during, and after applying the formulation), total biomass measurements of plants, root growth determinations, and overall health monitoring of plants. In particular, in vitro assays can be conducted to test the efficacy of the formulation in the presence of pathogens. In one instance, the colony growth inhibition assay includes contacting the formulation with a pathogen colony and measuring the diameter of pathogen colonies at various time points, thereby providing percentage inhibition as compared to a negative control (e.g., a colony contacted with water or with the formulation components but lacking the Trichoderma species). In another instance, the conidial germination inhibition assay includes contacting the formulation with a pathogen colony and measuring germination based on whether germ tube elongation is present or absent, thereby providing a percentage inhibition based on the observation of such presence or absence in 100 randomly selected colonies. Non-limiting characteristics of formulations include a colony growth inhibition of more than about 80% and/or a conidial germination inhibition of more than about 75%.

Such tests can be conducted to determine the shelf life of the formulation. For example, viability assays can be conducted before and after storage (e.g., any storage period, such as 1, 2, 3, 4 months, or longer). In one instance, an aliquot of the formulation can be prepared with growth media, which can then be pipetted to moisten a sterile disc of filter paper. Three technical replicates can be prepared for each individual sample, and each filter disc can 1 be placed opposite an agar dish. Cultures will be incubated for four days at 28° C. in the dark, and the overall colonization potential for each formulation can be assessed as the total surface area of the agar dish colonized by Trichoderma. In another instance, tests can include counting the number of spores to determine the CFU per mL before and after the storage period.

### Methods of Using Formulations

Methods of using the formulation can include treating a plant, such as by preparing a composition including any formulation herein and delivering the composition to a plant, a portion thereof, a plant material, or a soil in proximity to the plant. The composition can include only the formulation. In another embodiment, the composition can include the formulation (e.g., any described herein) and an aqueous solvent (e.g., water). Such methods can be useful for, in some instances, in preventative treatment of plants or plant materials.

The formulations herein can be applied or delivered to a plant in any useful manner. Delivering can include any useful method, such as by providing the composition by way of fertigation delivery to an irrigation system (e.g., a drip irrigation system), or by way of foliar delivery directly to plants. In one embodiment, the formulation can be applied by dissolving the formulation in an aqueous solvent prior to delivery to the soil, the plant, or the plant material (e.g., seed, seedling, bulb, etc.). In another embodiment, application can include pilling the seeds of a plant (e.g., using any coating described herein). In yet another embodiment, application can include immersion of the plant or the plant material in a bath containing a formulation herein. Furthermore, application can include providing one or more additives (e.g., any herein) before, during, or after delivery of the formulation to the soil, the plant, or the plant material. Other modes of delivery include broadcast application, liquid or dry in-furrow application, spraying, atomizing, vaporizing, misting, scattering, dusting, coating, watering, squirting, sprinkling, pouring, fumigating, and the like to the locus to be protected.

In particular embodiments, delivery can result in treating a plant. Such treating can include protecting the plant against a pathogenic organism, stimulating growth of the plant, or counteracting growth of a pathogenic organism in proximity to the plant. Use can include delivering an effective amount of the formulation onto the soil and/or on the plants for such treatment. Such delivery can include drip irrigation (e.g., at or close to transplanting, with optional delivery at a later interval after 14-30 days), spraying (e.g., directed either in furrow during planting or to the soil surface after planting), soaking (e.g., soaking of soil; and/or soaking of plants or plant materials prior to planting), soil treatment (before or after sowing or transplanting), direct mixing with soil, direct application to seeds, and the like. Such treatment can provide, for instance, better root development, increased proliferation of secondary roots, increased seedling weight, and/or improved crop yields.

Delivery or treatment can include any useful regime. In one instance, delivery includes providing the formulation at or close to transplanting. In another instance, delivery can include application at one or more stages of plant growth (e.g., germination, flowering, fruiting, etc.). Application can include one or more locations on the plant or soil (e.g., stem, leaves, roots, flowers, and/or fruit). Such application can include multiple applications to a single plant at different times (e.g., at or close to transplanting, with optional delivery at a later interval after 14-30 days). In another instance, such delivery or treatment can facilitate displacement of pathogenic microorganism by implanting Trichoderma species in the soil.

The formulations herein can be employed in any useful plant, portion thereof (e.g., roots, tubers, stems, flowers, and/or leaves), plant material (e.g., seed, seedlings, bulbs, etc.), or soil for growing such plants. Non-limiting plants include crop plants, turfs, ornamentals, agronomic row or other field crops, plants prone to fungal contamination (e.g., tomatoes, bell peppers, soy, and the like), and plants prone to new pathogenic fungi (e.g., rice, wheat, and the like). Yet other plants include agricultural plant species, including coffee, cucumber, tomato, pepper, and radish.

In some embodiments, the formulations herein can be used against pathogens, including pathogenic fungi. Non-limiting pathogens include Botryosphaeria (e.g., B. dothidea), Botrytis (e.g., B. cinerea), Clarireedia (e.g., C. homoeocarpa), Colletotrichum (e.g., C. coccodes), Fusarium (e.g., F. oxysporum), Macrophomina (e.g., M. phaseolina), Phytophthora (e.g., P. cactorum, P. cinnamomi, P. citricola, P. citrophthora, P. cryptogea, P. drechsleri, P. infestans, and/or P. nicotianae), Pythium (e.g., P. aphanidermatum, P. irregulare, P. spiculum, and/or P. ultimum), Rhizoctonia (e.g., R. solani), Rosellinia (e.g., R. necatrix), Sclerotinia (e.g., S. homoeocarpa, S. sclerotiorum, and/or S. solfsii), Sclerotium (e.g., S. rolfsii), Thielaviopsis (e.g., T. basicola) species, Verticillium (e.g., V. dahliae), as well as combinations thereof.

### EXAMPLES

### Example 1: Formulation 1 Including Trichoderma

Formulation 1 can include a plurality of Trichoderma species. A non-limiting example of Formulation 1 can be a solid formulation including the following:

| Component | Concentration | Type |
|---|---|---|
| T6 spores | 0.05 to 20% (w/w) | T. parareesei isolate |
| T59 spores | 0.05 to 20% (w/w) | T. virens isolate |
| Kaolin, | 80 to 99% (w/w) | Solid carrier (coating |
| sorbitol, | | surrounding particle |
| and/or sugar | | and spores) |

In one embodiment, the T6 and T59 spores are reproduced on rice kernels and maintained at about 4° C. Each kernel is coated with T6 spores, T59 spores, or a combination of T6 and T59 spores. Optionally, the spores can then be separated from the kernels prior to applying the solid carrier. This carrier can be, for instance, kaolin, sorbitol, and/or sugar.

In another embodiment, the formulation can include a solid particle having an inner core of a solid carrier (e.g., a rice kernel or any described herein), in which each particle is then coated with T6 spores, T59 spores, or a combination of T6 and T59 spores. In this way, the formulation can include particles just including T6 spores, which can be mixed with other particles having T59 spores. Alternatively, the formulation can include particles in which each particle includes a mixture of T6 and T59 spores. Then, the resulting particles can be coated with a coating to form a solid carrier. This carrier can be, for instance, kaolin, sorbitol, and/or sugar.

In use, Formulation 1 can be dispersed in an aqueous solvent (e.g., water) at any useful ratio for use as a foliar spray. In another use, Formulation 1 can be added to an irrigation system at any useful concentration for fertigation use. Optionally, Formulation 1 can be used with one or more biostimulants (e.g., any described herein, such as Formulations 5 and 6). Non-limiting concentrations can include an application of about 1 to 3 kilograms per hectare (kg/ha) nun a growing cycle, as well as multiple applications within a single cycle (e.g., a first application of about 1 kg/ha, a second application of about 0.5 kg/ha, and a third application of about 0.5 kg/ha). Other application rates and concentrations within one or more cycles are also encompassed by this disclosure. The dose can depend on the sanitary state of the soil where it is going to be applied.

### Example 2: Formulation 2 Including Trichoderma

Formulation 2 can include a plurality of Trichoderma species. A non-limiting example of Formulation 2 can be a solid formulation including the following:

| Component | Concentration | Type |
|---|---|---|
| T6 spores | 0.05 to 20% (w/w) | T. parareesei isolate |
| T59 spores | 0.05 to 20% (w/w) | T. virens isolate |
| T11 spores | 0.05 to 20% (w/w) | T. atroviride isolate |
| T25 spores | 0.05 to 20% (w/w) | T. asperellum isolate |
| Kaolin, | 20 to 98% (w/w) | Solid carrier (coating |
| sorbitol, | | surrounding particle |
| and/or sugar | | and spores) |

In one embodiments, the T6, T59, T11, and T25 spores are reproduced on rice kernels and maintained at about 4° C. Each kernel is coated with T6 spores, T59 spores, T11 spores, T25 spore, or a combination of any of these. Optionally, the spores can then be separated from the kernels prior to applying the solid carrier. This carrier can be, for instance, kaolin, sorbitol, and/or sugar.

In another embodiment, Formulation 2 includes a solid particle having an inner core of a solid carrier (e.g., a rice kernel or any described herein), in which each particle is coated with T6 spores, T59 spores, T11 spores, T25 spore, or a combination of any of these. This particle is then coated with a coating formed of a solid carrier. This carrier can be, for instance, kaolin, sorbitol, and/or sugar.

In use, Formulation 2 can be dispersed in an aqueous solvent (e.g., water) at any useful ratio for use as a foliar spray. In another use, Formulation 2 can be added to an irrigation system at any useful concentration for fertigation use. Non-limiting application rates and concentrations are provided in Example 1. Optionally, Formulation 2 can be used with one or more biostimulants (e.g., any described herein, such as Formulations 5 and 6).

### Example 3: Formulation 3 Including Trichoderma

Formulation 3 can include a plurality of Trichoderma species in a liquid formulation. A non-limiting example of Formulation 3 can include the following:

| Component | Concentration | Type |
|---|---|---|
| T6 spores | 0.05 to 20% (w/w) | T. parareesei isolate |
| Cellulose | 80 to 99.5% (w/w) | Solid carrier (coating |
| | | surrounding particle |
| | | and spores) |
| Water | — | Liquid carrier |

In one embodiment, Formulation 3 includes T6 spores and a coating formed of a solid carrier. This carrier can be, for instance, cellulose. In another embodiment, Formulation 3 includes a solid particle having an inner core of a solid carrier (e.g., a rice kernel or any described herein), in which each particle is coated with T6 spores. This particle is then coated with a coating formed of a solid carrier. This carrier can be, for instance, cellulose.

The formulation can be prepared with a solvent, e.g., water, at any useful concentration. Non-limiting concentrations include about 0.01 to about 0.1 kg of the spores per hectoliter of solvent.

If desired, one or more additional Trichoderma species can be included in the formulation. Additional species can be included by way of mixing the T6 spores with spores of other Trichoderma species in forming particles, as well as by introducing other populations of particles having spores of other Trichoderma species.

In use, Formulation 3 can be dispersed in an aqueous solvent (e.g., water) at any useful ratio for use as a foliar spray. In another use, Formulation 3 can be added to an irrigation system at any useful concentration for fertigation use. Non-limiting application rates and concentrations are provided in Example 1. Optionally, Formulation 3 can be used with one or more biostimulants (e.g., any described herein, such as Formulations 5 and 6).

### Example 4: Formulation 4 Including Trichoderma

Formulation 4 can include a plurality of Trichoderma species in a liquid formulation. A non-limiting example of Formulation 4 can include the following:

| Component | Concentration | Type |
|---|---|---|
| T6 spores and | 0.1 to 99.9% (v/v) | T. parareesei isolate |
| mycelium | | |
| T59 spores and | 0.1 to 99.9% (v/v) | T. virens isolate |
| mycelium | | |
| Mineral oil | 0 to 99.8% (v/v) | Liquid carrier |

As can be seen, Formulation 4 includes both spores and mycelium of the Trichoderma species. Furthermore, the liquid formulation can be a concentrate (with minimal or no liquid carrier) or diluted formulation having a liquid carrier.

In use, Formulation 4 can be dispersed in an aqueous solvent (e.g., water) at any useful ratio for use as a foliar spray. In another use, Formulation 4 can be added to an irrigation system at any useful concentration for fertigation use. Non-limiting application rates and concentrations are provided in Example 1. Optionally, Formulation 4 can be used with one or more biostimulants (e.g., any described herein, such as Formulations 5 and 6).

### Example 5: Formulation 5 Including Biostimulants

Formulation 5 can include biostimulants in a solid formulation, which can be used in combination with a Trichoderma formulation. A non-limiting example of Formulation 5 can include the following:

| Component | Concentration | Type |
|---|---|---|
| Proline | 5 to 95% (w/w) | Biostimulant |
| | | (amino acid) |
| Kaolin | 5 to 95% (w/w) | Solid carrier |
| Kelp | 0 to 90% (w/w) | Solid carrier |

As can be seen, Formulation 5 includes a biostimulant and a solid carrier. Optionally two or more solid carriers can be present.

In use, Formulation 5 can be dispersed in an aqueous solvent (e.g., water) at any useful ratio for use as a foliar spray. In another use, Formulation 5 can be added to an irrigation system at any useful concentration for fertigation use. Optionally, Formulation 5 can be used with one or more formulations including Trichoderma species (e.g., any described herein, such as Formulations 1-4).

### Example 6: Formulation 6 Including Biostimulants

Formulation 6 can include biostimulants in a liquid formulation as a suspension, which can be used in combination with a Trichoderma formulation. A non-limiting example of Formulation 6 can include the following:

| Component | Concentration | Type |
|---|---|---|
| Glutamic acid | 5 to 30% (w/w) | Biostimulant |
| and glutamine | | (amino acid) |
| Lysine | 5 to 30% (w/w) | Biostimulant |
| | | (amino acid) |
| Dihydrophaseic | at least 2000 | Biostimulant |
| acid | pmol/ml | (carotenoid) |
| Boron | 0.02 to 5% (w/v) | Biostimulant |
| | | (micronutrient) |
| Water | 40 to 70% (v/v) | Liquid carrier |

As can be seen, Formulation 6 includes a plurality of biostimulants and a liquid carrier. Additional biostimulants and additives may be included, if desired.

In use, Formulation 6 can be dispersed in an aqueous solvent (e.g., water) at any useful ratio for use as a foliar spray. In another use, Formulation 6 can be added to an irrigation system at any useful concentration for fertigation use. Optionally, Formulation 6 can be used with one or more formulations including Trichoderma species (e.g., any described herein, such as Formulations 1-4).

### Other Embodiments

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth, and follows in the scope of the claims.

Other embodiments are within the claims.

```
SEQUENCES
GenBank Accession No.: AJ251698, Trichoderma reesei
internal transcribed spacer 1 (ITS1), isolate 6
(SEQ ID NO: 10):
    1 ccgagtctac aactcccaaa ccccaatgtg aacgttacca atctgttgcc tcggcgggac
  61 tctctgcccc gggcgcgtcg cagccccgga tcccatggcg cccgccggag gaccaactca
121 aactcttctc cccctccgtc gcggcttccg tcgcggctct gtctcaccct tgctctgagc
161 ctttctcggc gaccctagcg ggcgtctcga aaatgaatca
GenBank Accession No.: AJ563621, Hypocrea jecorina
partial tef1 gene for translation elongation
factor 1, exons 5-6, strain IMI 113135
(SEQ ID NO: 11):
    1 cccaagtact atgtcaccgt cattggtatg ttggcagcca acatctcatt gcgtcgtcga
  61 cacgtcaaac taacgatgcc ctcacagacg ctcccggcca ccgtgactcc atcaagaaca
121 tgaccaccgg cacttcccag g
GenBank Accession No.: KF699130, Trichoderma parareesei
strain T6 translation elongation
factor 1-alpha (lefl) gene, partial cds (SEQ ID NO: 12):
    1 attctccctt gcctatctgt ccaacatttg tcgaccaaat gttgcgccga caggtttttt
  61 tcatcacccc gccttcttct acccctccga gcgacgcaaa ctcttttgct gccttacgat
121 gggttttagt ggggttgcat cgagcaaccc caccaatact ctggccgctc tgccggatcc
181 ttcgacaaca gtcacctcag cacacgcgtc accaacacag cagtctttga tccgcgatgc
241 taaccatgtt cccctccata ggaagccgcc gaactcggca agggttcctt caagtacgcg
301 tgggttctcg acaagctcaa ggccgagcgt gagcgtggca tcaccatcga cattgccctc
361 tggaagttcg agactcccca gtactatgtc accgtcattg gtatgttggc agccaacatc
421 tcattgcgtc gttgacacgt caaactaacg atgccctcac agacgctccc ggccaccgtg
481 acttcaccaa gaacatgatc actggtactc cccaggccga ctgcgctatc ctcattaccg
541 ctgccggtac tggtgagttc gaggctggta tctccaagga tggccagacc cgtgagcacg
601 ctctgctcgc ctacaccctg ggtgtcaagc agctcatcgt cgccatcaac aagatggaca
861 ctgccaactg ggccgaggct cgttaccagg aaatcatcaa ggagacttcc aacttcatca
721 agaaggtcgg cttcaacccc aaggccgttg cttcc
GenBank Accession No.: KF699131, Trichoderma parareesei
strain T6 calmodulin (CALI)
gene, partial cds (SEQ ID NO: 13):
    1 ggggggttgt ttacagggtg ctgaccgagc tgctctccag gacaaggacg gcgatggtac
  61 gtgatggcga gtgacgcgac aacacactta ttgccctctc tacgaagccg caccgaagca
121 ctttttgccg atcgatcact ctctcgtcga ctcgaateat gatacatgga caagaaactg
181 acaggcttga cctcgtaggc cagatcacca ccaaggagct gggcaccgtg atgcgctctc
241 ccggccagaa cccctccgag tcggagctgc aggacatgat caacgaggtt gacgccgaca
301 acaacggttc categacttc cctggtacgc gaattgttgg gagatttggt ggttgaggta
361 cacgggctga cgtggagcgg tgaagaacgg ctcacca
Primer EF1-728F for tef1 (SEQ ID NO: 14):
5′-CATCGAGAAGTTCGAGAAGG-3′
Primer TEFl-LLErev for tef1 (SEQ ID NO: 15):
5′-AACTTGCAGGCAATGTGG-3′
Primer CAL-228F for cal1 (SEQ ID NO: 16):
5′-GAGTTCAAGGAGGCGTTCTCCC-3′
Primer CAL-737R for cal1 (SEQ ID NO: 17):
5′-CATCTTTCTGGCCATCATGG-3′
GenBank Accession No.: AJ517317, Trichoderma virens
internal transcribed spacer 1 (ITS 1)
(SEQ ID NO: 20):
    1 ccgagtttac aactcccaaa cccaatgtga sogttaccaa actgttgcct cggcgggatc
  61 tctgccccgg gtgcgtcgca gccccggacc aaggcgcccg ccggaggacc aaccaaaact
121 cttattgtat accccctcgc gggtttttta ctatctgagg catctcggcg cccctcgtgg
GenBank Accession No.: FJ788527.1, Hypocrea virens
strain T59 thioredoxin-like protein (Dim 1)
gene, complete cds (SEQ ID NO: 21):
    1 atgggctctg tcgttctccc gcatctaaac tcaggctggc acgccgacca ggccatttta
  61 tcgggttcgt cactctcctt gccgttgctc catcttattt atcgtgacac gtgatgcagt
121 gtgtgctgac cagttgcata gaagaggatc gtctcgtcgt catccggttc ggacgtgacc
181 acgatcgggt aagaagagtc acttttactc ttggatattt tcccttacta actgccatta
241 ggaccgcatg cLgcaagatg aagtgctcta caagatagcc gatcgcgtca aaaactttgc
301 cgtcatttac ctctgcgaca ttgacgaggt aggtttttca tctcatccat ggagtaaagg
361 ccgcttttaa ctggaagtag gctcctgatt tcaacgccat gtacgaaccg tacgaccctt
421 gctctattcc gttcttcttt cgcaacaagc atatgatgtg cgattttggt accggtaaca
481 acaacaagct taactgggtg ctggaggata agcaagagct cattgacatt attgaaacga
541 tctaccgcgg agcaaagaaa ggtagaggct tggcggtcag ccccaaggat cacagcacga
601 gacacagata ctag
GenBank Accession No.: ACY01406.1, thioredoxin-like
protein [Trichoderma virens], amino
acids 1-143 (SEQ ID NO: 22):
    1 MGSVVLPHLN SGWHVDQAIL SEEDRLVVIR FGRDHDRDCM LQDEVLYKIA DRVKNFAVIY
  61 LCDIDEVPDF NAMYELYDPC SILFFFRNKH MMCDFCTCNN NKLMWVLEDK QELIDIIETI
121 YRGAKKGRGL VVSPKDYSTR KPY
GenBank Accession No.: ACY01406.1, thioredoxin-
like protein [Trichoderma virens], amino
acids 6-137 (SEQ ID NO: 23):
    1 LPHLNSGWHV DQAILSEEDR LVVIRFGPDH DRDCMLQDEV LYKIADRVKN FAVIYLCDID
  61 EVPDFNAMYE LYDPCSILFF FRNKHMMCDF GTGRNNKLNW VLEDKQLEID IIETIYRGAK
121 KGRGLVVSPK DY
Primer TRX-5 for dim1 (SEQ ID NO: 24):
5′-GAAGAGGATCGTCTCGTCGTC-3′
Primer TRX-3 for dim1 (SEQ ID NO: 25):
5′-TCAGGAACCTCGTCAATGTCG-3′
GenBank Accession No.: AJ224008,Trichoderma harzianum
5.8S rRNA and ITS1 and ITS2 DNA, isolate 11 (SEQ ID NO: 30):
    1 agggatcatt accgagttta caactcccaa acccaatgtg aaccatacca aactgttgcc
  61 tcggcggggt cacgccccgg gtgcgtcgca gccccggaac caggcgcccg ccggagggac
121 caaccaaact cttctctgta gtcccctcgc ggacgttatt tcttacagct ctgagcaaaa
181 attcaaaatg aatcaaaact ttcaacaacg gatctcttgg ttctggcatc gatgaagaac
241 gcagcgaaat gcgataagta atgtgaattg cagaattcag tgaatcatcg aatctttgaa
301 cgcacattgc gcccgccagt attctggcgg gcatgcctgt ccgagcgtca tttcaaccct
361 cgaacccctc cggggggtcg gcgttgggga cctcgggagc ccctaagacg ggatcccggc
421 cccgaaatac agtggcggtc tcgccgcagc ctctcctgcg cagtagtttg cacaactcgc
461 accgggagcg cggcgcgtcc acgtccgtaa aacacccaac ttctgacctg ttgacctcgg
542 atcaggtagg aatacccgct gaacttaa
GenBank Accession No.: AJ563609, Trichoderma cf. viride
partial tef1 gene for translation elongation factor 1,
exons 5-6, strain IMI 352941 (SEQ ID NO: 31):
    1 cccaagtact atgtcaccgt cattggtatg ttttcgcttt ccctcattga tacttggaga
  61 ccaagattct aacgtgccgc tctgtagacg ctcccggtca ccgtgatttc atcaagaaca
121 tgatcactgg tacttcccag g
GenBank Accession No.: AJ223773, Trichoderma viride
5.8S rRNA gene, ITS1 and ITS2, isolate 25 (SEQ ID NO: 40):
    1 agggatcatt accgagttta caactcccaa acccaatgtg aacgttacca aactgttgcc
  61 tcggcggggt cacgccccgg gtgcgtcgca gccccggaac caggcgcccg ccggaggaac
121 caaccaaact ctttctgtag tcccctcgcg gacgtatttc tttacagctc tgagcaaaaa
181 ttcaaaatga atcaaaactc tcaacaacgg atctctcggt tctggcatcg atgaagaacg
241 cagcgaaatg cgataagcaa tgtgaattgc agaattcagt gaaccatcga atctttgaac
301 gcacattgcg cccgccagta ttctggcggg catgcccgtc cgagcgtcat ttcaaccctc
361 gaacccctcc gggggatcgg cgttggggat cgggacccct cacacgggtg ccggccccta
421 aatacagtgg cggtctcgcc gcagoctctc ctgcgcagta gtttgcacaa ctcgcaccgg
481 gagcgcggcg cgtccacgtc cgtaaaacac ccaactttct gaaatgttga cctcggatca
341 ggtaggaata cccgctgaac ttaa
GenBank Accession No.: AJ563611, Trichoderma asperellum
partial tef1 gene for translation elongation factor 1,
exons 5-6, strain IMI 296237 (SEQ ID NO: 41):
    1 cccaagtact atgtcaccgt cattggtatg ttttggactc ttctctctag ctatcgacat
  61 tccaagtccg ccattctaac atgctcttcc cacagacgct cccggtcacc gtgatttcat
121 caagaacatg atcactggta cctcccagg
5′ primer binding to the 3′ end of the 18S rDNA gene and
in proximity to the 5′ end of the ITS1 region
(ITS1 primer, SEQ ID NO: 42):
5′-TCCGTAGGTGAACCTGCGG-3′
3′ primer binding to the 5′ end of the 5.8S rDNA gene and
in proximity to the 3′ end of the ITS1 region
(ITS2 primer, SEQ ID NO: 43):
5′-GCTGCGTTCTTCATCGATGC-3′
3′ primer binding to the 5′ end of the 28S rDNA gene and
in proximity to the 3′ end of the ITS2 region
(ITS4 primer, SEQ ID NO: 44):
5′-TCCTCCGCTTATTGATATGC-3′
5′ primer binding to the 5′ end of the 5.8S rDNA gene
(ITS3 primer, SEQ ID NO: 45):
GCATCGATGAAGAACGCAGC-3′
Primer tef1fw for tef1 (SEQ ID NO: 46):
5′-GTGAGCGTGGTATCAGCA-3′
Primer tef1 rev for tef1 (SEQ ID NO: 47):
5′-GCCATCCTTGGAGACCAGC-3′
GenBank Accession No.: AF278790, Trichoderma harzianum
strain CECT 2413 internal transcribed spacer 1, partial
sequence; 5.8S ribosomal RNA gene and internal
transcribed spacer 2, complete sequence; and 28S ribosomal
RNA gene, partial sequence (SEQ ID NO: 50):
    1 accgaattta caactcccaa acccaatgtg aacgttacca aagtgtcgcc tcggcgggat
  61 ctctgacccc gggtgcgtcg cagccccgga ccaaggcgcc cgccgganga ccaaccaaaa
121 ctcttattgt ataccccctc gcgggttttt tttataatct gagccttctc ggcgcctctc
181 gtaggcgttt cgaaaatgaa tcaaaacttt caacaacgga tctcttggtt ctggcatcga
241 tgaagaacgc agcgaaacgc gataagtaat gtgaattgca gaattcagtg aatcatcgaa
301 tctttgaacg cacattgcgc ccgccagtat tttggcgggc atgcctgtcc gagcgtcatt
361 tcaaccctcg aacccctccg gggggtcggc gtcggggatc ggccctgcct tggcggtggc
421 cgctcccgaa atacagtggc ggtttcgccg cagcctttcc tgcgcagtag tttgcacact
481 cgcatcggga gcgcggcgcg tccacagccg ttaaacaccc aacttctgaa atgttgacct
541 cggat
GenBank Accession No.: FJ545255, Hypocrea lixii
strain ATCC 20847 18S ribosomal RNA gene, partial
sequence; internal transcribed spacer, 5.8S ribosomal
RNA gene, and internal transcribed spacer 2, complete
sequence; and 28S ribosomal RNA gene, partial sequence
(SEQ ID NO: 51):
    1 gcggagggat cattaccgag tttacaactc ccaaacccaa tgtgaacgtt accaaactgt
  61 cgcctcggcg ggatctctgc cccgggtgcg tcgcagcccc ggaccaaggc gcccgccgga
121 ggaccaacca aaactcttat tgtatacccc ctcgcgggtt tttttataat ctgagccttc
181 tcggcgcctc tcgtaggcgt ttcgaaaatg aatcaaaact ttcaacaacg gatctcttag
241 ttctggcatc gatgaagaac gcagcgaaat gcgataagta atgtgaattg cagaattcag
301 tgaatcatcg aatctttgaa cgcacattgc gcccgccagt attctggcgg gcatgcctgt
361 ccgagcgtca tttcaaccct cgaacccctc cggggggtcg gcgttgggga tcggccctgc
421 cttggcggtg gccgtctccg aaatacagtg gcggtctcgc cgcagcctct cctgcgcagt
481 agtttgcaca ctcgcatcgg gagcgcggcg cgtccacagc cgttaaacac ccaacttctg
341 aaatgttgac ctcggatcag gtaggaatac ccgctgaact taagcatatc a
GenBank Accession No.: KU933430, Trichoderma afroharzianum
strain ATCC 20847 translation elongation factor 1-alpha (EF1a)
gene, partial cds (SEQ ID NO: 52):
    1 cactggtact tcccaggccg attgcgctat cctcatcatt gccgccggta ctggtgagtt
  61 cgaggctggt atctccaagg atggccagac ccgtgagcac gctctgctcg cctacaccct
121 gggtgttaag cagctcatcg ttgccatcaa caagatggac actgccaact gggccgaggc
181 tcgttaccag gaaatcatca aggagacttc caacttcatc aagaaggtcg gcttcaaccc
241 caaggctgtt gctttcgtcc ccatctccgg tttcaacggt gacaacatgc tccagccctc
301 caccaactgc ccctggtaca agggctggga gaaggagacc aaggctggca agttcaccgg
361 caagaccctc cttgaggcca tcgactccat cgagcccccc aagcgtccca cggacaagcc
421 cctccgtctt cccctccagg atgtctacaa gatcggtggt attggaacag ttcccgtcgg
481 ccgtatcgag actggtgtcc tcaagcccgg tatggtcgtc actttcgctc cctccaacgt
541 caccactgaa gtcaagtccg ccgagatgca ccacgagcag ctcgtcgagg gtgttcccgg
601 cgacaacgtt ggtttcaacg tcaagaacgt ttccgttaag gaaattcgcc gtggtaacgt
661 tgccggtgac tccaagaacg acccccccat gggtgccgct tctttcaccg ctcaggtcat
721 cgtcatgaac caccctggcc aggtcggtgc cggctacgcc cccgttcttg actgccacac
781 tgcccacatt gcctgcaagt tcgccgagct ccaggagaag atcgaccgcc gtaccggtaa
841 ggctaccgag actgccccca agttcatcaa gtccggtgac tctgccatcg tcaagatgat
901 tccctccaag cccatgtgcg ttgaggcttt caccgactac cctcccctgg gtcgtttcgc
961 cgtccgtg
GenBank Accession No.: AJ251702, Trichoderma longibrachiatum
internal transcribed spacer 1 (ITS 1), isolate F724 (SEQ ID NO: 53):
    1 ccgagtttac aactcccaaa cccaatgtga acgttaccaa tctgttgcct cggcgggatt
  61 ctcttgcccc gggcgcgtcg cagccccgga tcccatggcg cccgccggag gaccaactcc
121 aaactctttt tttctctccg tcgcggctcc cgtcgcggct ctgttttatt tttgctctga
181 gcctttctcg gcgaccctag cgggcgtctc gaaaatgaat ca
```

1. A formulation comprising a Trichoderma parareesei isolate and a Trichoderma virens isolate.

2. The formulation of claim 1, wherein the Trichoderma parareesei isolate is T. parareesei strain T6 and the Trichoderma virens isolate is T. virens strain T59.

3. The formulation of claim 1, further comprising an isolate of Trichoderma atroviride and/or an isolate of Trichoderma asperellum.

4. The formulation of claim 3, wherein the isolate of Trichoderma atroviride is T. atroviride strain T11 and/or the isolate of Trichoderma asperellum is T. asperellum strain T25.

5. The formulation of claim 1, wherein the formulation further comprises a liquid carrier, and wherein the isolates comprise mycelium and/or spores of T. parareesei and T. virens.

6. The formulation of claim 5, wherein the liquid carrier comprises a mineral oil having one or more optional stabilizers.

7. The formulation of claim 1, wherein the formulation further comprises a solid carrier, and wherein the isolates comprise spores of T. parareesei and T. virens.

8. The formulation of claim 7, wherein the solid carrier comprises a water-soluble, a wettable, or a water-dispersible particle, and wherein the spores are disposed on or within the particle.

9. The formulation of claim 8, wherein the particle comprises a clay, a sugar alcohol, a sugar, a saccharide, or a polysaccharide.

10. The formulation of claim 8, wherein the particle is a powder, a pellet, or a granule.

11. The formulation of claim 1, wherein a concentration of the isolates is from about 0.1 to 5% (w/w) of a solid formulation.

12. The formulation of claim 1, wherein a concentration of each isolate is from about 1×10⁷ to 1×10¹⁰ colony-forming units per gram (CFU/g) of a solid formulation.

13. The formulation of claim 1, wherein a concentration of the isolates is from about 0.1 to 100% (w/v) or (v/v) of a liquid formulation.

14. The formulation of claim 1, wherein a concentration of each isolate is from about 1×10⁵ to 1×10⁸ spores per milliliter of a liquid formulation.

15. The formulation of claim 1, further comprising one or more metabolic inhibitors, stabilizers, nutrients, or additives.

16. The formulation of claim 1, further comprising a biostimulant, wherein the biostimulant is separated from the isolates.

17. A formulation comprising:
one or more Trichoderma isolates (e.g., cellulose coated spheres);
a solid carrier; and
a liquid carrier (e.g., water).

18. The formulation of claim 17, wherein the one or more Trichoderma isolates comprise Trichoderma parareesei, Trichoderma vixens, Trichoderma atroviride, and/or Trichoderma asperellum.

19. The formulation of claim 17, wherein the one or more Trichoderma isolates comprise a plurality of spores of Trichoderma.

20. The formulation of claim 19, further comprising a coating surrounding at least one of the plurality of spores.

21. The formulation of claim 20, wherein the coating comprises a polysaccharide (e.g., cellulose).

22. The formulation of claim 17, wherein the solid carrier comprises a pellet.

23. The formulation of claim 17, wherein the liquid carrier comprises an aqueous solvent or an oil.

24. The formulation of claim 17, further comprising one or more metabolic inhibitors, stabilizers, nutrients, or additives.

25. The formulation of claim 17, further comprising a biostimulant, wherein the biostimulant is separated from the isolates.

26. A binary formulation comprising:
a first formulation comprising the formulation of claim 1; and
a second formulation comprising one or more biostimulants, wherein the first and second formulations are configured to be separated and then to be combined for delivery to a plant.

27. The binary formulation of claim 26, wherein the first formulation comprises a liquid formulation, a concentrated liquid formulation, or a solid formulation; and wherein the second formulation comprises a liquid formulation, a concentrated liquid formulation, or a solid formulation.

28. A method of treating a plant, the method comprising:
preparing a composition comprising the formulation of claim 1 and an aqueous solvent; and
delivering the composition to the plant, a portion thereof, a plant material, or a soil in proximity to the plant.

29. The method of claim 28, further comprising, prior to said delivering:
providing the composition to an irrigation system configured to irrigate the plant.

30. The method of claim 28, wherein said treating comprises protecting the plant against a pathogenic organism, stimulating growth of the plant, or counteracting growth of a pathogenic organism in proximity to the plant; and wherein said delivering comprises delivering an effective amount of the composition for said treating.

31. A binary formulation comprising:
a first formulation comprising the formulation of claim 17; and
a second formulation comprising one or more biostimulants, wherein the first and second formulations are configured to be separated and then to be combined for delivery to a plant.

32. The binary formulation of claim 31, wherein the first formulation comprises a liquid formulation, a concentrated liquid formulation, or a solid formulation; and wherein the second formulation comprises a liquid formulation, a concentrated liquid formulation, or a solid formulation.

33. A method of treating a plant, the method comprising:
preparing a composition comprising the formulation of claim 17 and an aqueous solvent; and
delivering the composition to the plant, a portion thereof, a plant material, or a soil in proximity to the plant.

34. The method of claim 33, further comprising, prior to said delivering:
providing the composition to an irrigation system configured to irrigate the plant.

35. The method of claim 33, wherein said treating comprises protecting the plant against a pathogenic organism, stimulating growth of the plant, or counteracting growth of a pathogenic organism in proximity to the plant; and wherein said delivering comprises delivering an effective amount of the composition for said treating.
